(19) United States
(12) Patent Application Publication
(10) Pub. No.: US 2004/0186304 A1
(43) Pub. Date: Sep. 23, 2004

(54) Process for continuously preparing acetals of alpha, beta-dicarbonyl compounds

(75) Inventors: Johannes Ruwwe, Niederkassel (DE); Jutta Bruhl, Dorsten (DE); Clemens Osterholt, Dorsten (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/805,257

(22) Filed: Mar. 22, 2004

(30) Foreign Application Priority Data
Mar. 21, 2003 (DE) 103 12 562.0

(51) Int. Cl. C07D 323/02; C07C 43/32

(52) U.S. Cl. 549/430; 568/593

(57) ABSTRACT

The present invention relates to a process for preparing acetals of α,β-dicarbonyl compounds of the general formula

(R″O)₂CRCR′(OR″)₂

which are obtained by continuous reaction of α,β-dicarbonyl compounds R—CO—CO—R′ with alcohols R″OH or HO—X—OH in countercurrent.

### BACKGROUND OF THE INVENTION

1. Field of the Invention

The present invention relates to a process for continuously preparing acetals of α,β-dicarbonyl compounds.

2. Discussion of the Background

Acetals of α,β-dicarbonyl compounds of the (R″O)₂CRCR′(OR″)₂ (I) type, for example those of glyoxal, can be used as crosslinkers for fibrous materials made of polyvinyl alcohol or in the processing of cellulose, paper, leather and cotton. Such compounds can likewise be used as auxiliaries in the processing of ores.

Use of acetals of glyoxal as an additive in diesel and petroleum is described in DE 199 20 270.

A further application is specified in WO 0220446, which describes an electrochemical preparation of orthocarboxylic esters from acetals of α,β-dicarbonyl compounds.

Acetals of α,β-dicarbonyl compounds can be prepared by reacting α,β-dicarbonyl compounds with alcohols. These reactions are customarily acid-catalyzed.

R—CO—CO—R′+4R″OH→(R″O)₂CRCR′(OR″)₂+2 H₂O

For the example of glyoxal (R, R″═H), such processes have been described in batchwise mode, for example in U.S. Pat. No. 2,360,959, GB 559362, U.S. Pat. No. 2,360,957, U.S. Pat. No. 2,361,456, SU 434078, U.S. Pat. No. 5,191,127, and also in J. Org. Chem. (1972), 37, 1276; J. Org. Chem. (1973), 38, 556; J. Org. Chem. (1974), 39, 1772 and Recueil des Travaux Chimique des Pays-Bas (1990), 109, 15. The water of reaction which is formed, and the water which is used as a solvent, are removed by co-distilling with the alcohol used, or by azeotropic distillation with an additional substance (azeotroping agent).

The disadvantage of the processes described lies in the poor space-time yields, the sometimes moderate yields, especially when using low-boiling alcohols such as methanol or ethanol (R″=Me, Et) and sometimes unsatisfactory selectivity.

Similar reactions with higher homologous α,β-dicarbonyl compounds are described, for example, in Bull. Soc. Chim. Fr. (1976), 3-4, 601; and J. Chem. Soc., Perkin Trans. II (1972), 4, 357. U.S. Pat. No. 2,421,559 and EP 0704422 specify specific compounds I (R═H, R″=methyl, R″=methyl, butyl) as undesired by-products in the synthesis of methyl glyoxal dialkyl acetals (Me-CO—CH(OR″)₂.

The preparation of the acetals of α,β-dicarbonyl compounds of the type (R″O)₂CRCR′(OR″)₂ by reaction of α,β-dicarbonyl compounds with alcohols by acidic catalysis can be accelerated by adding dehydrating agents. However, this has the disadvantage that additional substances, for example trimethylsilyl chloride (Synthesis (1983), 203) or trimethyl-orthoformate (J. Org. Chem. (1996), 61, 3897), have to be additionally introduced into the process, which considerably reduces the economic viability of such a process.

A further method for preparing acetals of α,β-dicarbonyl compounds, especially glyoxyl tetraethylacetal (I; R, R′═H, R″=ethyl), is the reaction of ethanol with NOCI, as described in U.S. Pat. No. 3,130,234. The disadvantage of this process is that only this stated derivative is accessible and that nitrosyl chloride, which is of limited availability, has to be handled.

### SUMMARY OF THE INVENTION

It is therefore an object of the present invention to specify a process for preparing acetals of α,β-dicarbonyl compounds whose application, especially on the industrial scale, is advantageous from economic and ecological points of view, and which, in addition, avoids the disadvantages of the prior art processes.

### DETAILED DESCRIPTION OF THE INVENTION

This object is achieved by preparing, in a continuous process, compounds of the general formula (I)

(R″O)₂CRCR′(OR″)₂ (I),

where

R and R′ are each independently H, (C₁-C₈)-alkyl, (C₃-C₈)-cycloalkyl, (C₂—C₈)-alkenyl, (C₂-C₈)-alkynyl or (C₆-C₁₈)-aryl and

R″ is in each case independently (C₁-C₈-alkyl), (C₃-C₈)-cycloalkyl, (C₂-C₈)-alkenyl, (C₂-C₈)-alkynyl, or else a chain X where X is a (C₂-C₁₂)-alkylene chain or (C₂-C₁₂)-alkenylene chain which joins the two oxygen atoms of the αa-carbon atom and/or the two oxygen atoms of the β-carbon atom (acetals (Ia))

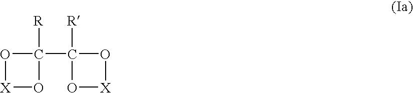

by reacting compounds of the type R—CO—CO—R′, where R and R′ are each as defined above with monohydric alcohols of the type R″OH or dihydric alcohols of the type HO—X—OH (glycols, diols), where R″ and X are each as defined above, in an apparatus which allows a continuous reaction by the countercurrent principle.

Surprisingly, the desired products can be isolated in good yields and purities with good space-time yields.

The invention therefore provides a process for preparing the compounds of the general formula (I) (R″O)₂CRCR′(OR″)₂.

The reaction is advantageously conducted in such a way that an α,β-dicarbonyl compound R—CO—CO—R′, either as a pure substance or in the form of a solution in water or an organic solvent, for example, an alcohol, amide, ester, ether, aliphatic, aromatic hydrocarbon or a halogenated hydrocarbon, is fed into a countercurrent apparatus, for example a reaction column, a thin-film evaporator, a falling-film evaporator or a stirred tank battery, while a monohydric alcohol R″OH or dihydric alcohol HO—X—OH, preferably in gaseous form, is simultaneouly fed into this apparatus in such a way that the ascending vapor of the alcohol R″OH or HO—X—OH flows in countercurrent to the descending α,β-dicarbonyl compound R—CO—CO—R′ or its solution in a solvent.

However, it is also possible to feed in the alcohol R″OH or HO—X—OH in liquid form and not to evaporate it until it is in the apparatus.

Depending on the volatility of the target compound (R″O)₂CRCR′(OR″)₂ and the boiling point of the alcohol used R″OH or HO—X—OH, the product is obtained with the distillate or with the bottom effluent of the apparatus used and can be purified by means of distillation.

The reaction can be effected in the presence or in the absence of a catalyst, preferably in the presence of a catalyst, in particular in the presence of a protic or Lewis acid. This catalyst can be added either in gaseous form with the monohydric alcohol R″OH or the dihydric alcohol HO—X—OH used, or else in dissolved form together with the α,β-dicarbonyl compound R—CO—CO—R′ used. Possible catalysts are, for example, sulfonic acids, sulfuric acid, hydrochloric acid, hydrogensulfates or carboxylic acids. It is likewise possible to use heterogeneous catalysts, for example ion exchangers, montmorillonites or acidic oxides, which are either secured to the wall of the apparatus or else introduced in the form of a catalytically active structured packing.

The reaction temperature is from 20 to 250° C., preferably from 50 to 200° C., more preferably from 60 to 150° C. It is possible to carry out the reaction without external cooling or heating, in which case the temperature is established via the boiling point of the alcohol R″OH or HO—X—OH used at the particular pressure and the temperature of the α,β-dicarbonyl compound R—CO—CO—R′, or its solution, flowing in countercurrent. It is likewise possible to cool or to heat the apparatus used. In addition, the streams fed can likewise be cooled or heated.

The reaction is carried out at pressures of from 20 mbar to 20 bar, preferably from 500 mbar to 5 bar. Particular preference is given to the pressure range from 1 to 3 bar.

The molar ratio in the streams introduced between the α,β-dicarbonyl compound R—CO—CO—R′ and the monohydric alcohol R″OH may be between 1:2 and 1:2000, and preference is given to ratios between 1:4 and 1:80, very particular preference to ratios between 1:8 and 1:20.

The molar ratio in the streams introduced between the α,β-dicarbonyl compound R—CO—CO—R′ and the dihydric alcohol HO—X—OH may be between 1:1 and 1:1000, and preference is given to ratios between 1:2 and 1:40, very particular preference to ratios between 1:4 and 1:10.

α,β-Dicarbonyl compounds used with preference are compounds of the general formula R—CO—CO—R′, in which R and R′ are each independently H, (C₁-C₈)-alkyl, (C₃-C₈)-cycloalkyl, (C₂-C₈)-alkenyl, (C₂-C₈)-alkynyl or (C₆-C₁₈)-aryl. However, preference is given to compounds in which R and R′ are each independently H or (C₁-C₈)-alkyl. Very particular preference is given to compounds in which R and R′ are H or methyl, R and R′ being independent of each other, thus in particular the compounds diacyl, glyoxal or methylglyoxal.

Alcohols used with preference are monohydric alcohols of the general formula R″OH in which R″ is (C₁-C₈-alkyl), (C₃-C₈)-cycloalkyl, (C₂-C₈)-alkenyl or (C₂-C₈)-alkynyl. The alcohols used may likewise be dihydric alcohols of the general formula HO—X—OH where X is a (C₂-C₁₂)-alkylene chain or a (C₂-C₁₂)-alkenylene chain. Especially preferred compounds are alcohols of the formula R″OH in which R″ is (C₁-C₈-alkyl), and very particular preference is given to methanol, ethanol, n-propanol, isopropanol, n-butanol, 2-butanol and 2-methyl-1-propanol. It is also possible to use mixtures of alcohols.

Depending on the physical properties of the products and reactants, the distillates and bottom effluents occurring in the process described comprise the following compounds in varying concentrations:

(R″O)₂CRCR′(OR″)₂ (I), (R″O)₂CR—CO—R′, R″OH and/or HO—X—OH, water, any unconverted α,β-dicarbonyl compound R—CO—CO—R′, in the case that R, R′═H (glyoxal), any oligomeric by-products, and also methyl 2-hydroxyacetate and 2-hydroxyacetic acid, from which the target compound can be isolated by continuous or batchwise distillation.

In order to be able to realize particularly economical processes, the alcohol R″OH or HO—X—OH and the hemiacetalized compound (R″O)₂CR—CO—R″ recovered from this distillation should be recycled into the process, in which case the alcohol R″OH or HO—X—OH is again fed into the apparatus used in gaseous form, and the hemiacetalized product (R″O)₂CR—CO—R′ is fed into the apparatus together with the α,β-dicarbonyl compound R—CO—CO—R′ in such a way that the countercurrent principle is maintained.

Preference is given to processes which are conducted in such a way that a monohydric alcohol R″OH or a dihydric alcohol HO—X—OH are metered in gaseous form at a lower inlet point of a vertical countercurrent apparatus, and, at the same time, at an upper inlet point to this apparatus, an α,β-dicarbonyl compound R—CO—CO—R′ is introduced without solvent or in solution in such a way that the resulting bottom effluent contains the desired acetal (R″O)₂CRCR′(OR″)₂ (I) and, where present, the hemiacetalized product (R″O)₂CR—CO—R′, and also the alcohol R″OH or HO—X—OH used, while the distillate consists only of the alcohol R″OH or HO—X—OH and water. The bottom effluent and distillate are worked up separately in further distillation apparatus. When sufficient purification is possible, the compounds (R″O)₂CR—CO—R′ and R″OH or HO—X—OH, in addition to the target product, are also obtained in very pure form and recycled into the process.

Very particular preference is given to processes in which not only the preparation of the product (R″O)₂CRCR′(OR″)₂ (I) but also the workup of the resulting bottom effluent and of the distillate are carried out by means of a continuous process.

Having generally described this invention, a further understanding can be obtained by reference to certain specific examples which are provided herein for purposes of illustration only, and are not intended to be limiting unless otherwise specified.

### EXAMPLES

### Example 1

### Preparation of 1,1,2,2-tetramethoxyethane

An aqueous glyoxal solution (40% by weight) which had been admixed with a small amount of a solution of p-toluenesulfonic acid in methanol was metered using a pump into the top of a heatable 1 m column (diameter 29 mm) with wire braid inserts. At the same time, gaseous methanol was fed in at the lowermost tray of the column. At the top of the column, a distillate mixture of water and methanol was collected, and a mixture of 1,1,2,2-tetramethoxyethane, 2,2-dimethoxyacetaldehyde and methanol was collected at the bottom discharge. The top distillate consisting of water and methanol was distilled in a further column, and the methanol was recycled, reevaporated and fed into the lowermost inlet of the column. The methanol can likewise be recovered in the reaction column when a further column which had a stream above the metering point of the glyoxal solution is used. The effluent mixture consisting of 1,1,2,2-tetramethoxyethane (TME), 2,2-dimethoxyacetaldehyde and methanol was likewise worked up by distillation in a further column, and the lower-boiling components methanol and 2,2-dimethoxyacetaldehyde were recycled into the process. Methanol was again evaporated and and fed in at the bottom of the column, and 2,2-dimethoxyacetaldehyde could be added to the aqueous glyoxal solution and fed in with it at the top of the column. The glyoxal and methanol were metered in at such a rate that the mole ratios of glyoxal to methanol present were from 1:4 mol up to 1:18. The column was either operated unheated or heated up to a temperature of 160° C.

TABLE 1
Yields of tetramethoxyethane obtained under a variety
of conditions.

| | | | Glyoxal | | |
|---|---|---|---|---|---|
| | | | metering | Reaction | |
| | Glyoxal:MeOH | Temp. | rate | time | TME Yield |
| No. | mole ratio | (° C.) | (mol/h) | (h) | (g (%)) |
| 1 | 1:4 | 90 | 0.17 | 8 | 112 (55%) |
| 2 | 1:8 | 90 | 0.17 | 8 | 138 (68%) |
| 3 | 1:12 | 100 | 0.46 | 4 | 226 (82%) |
| 4 | 1:12 | 90 | 0.25 | 8 | 276 (92%) |
| 5 | 1:18 | 100 | 0.17 | 8 | 161 (79%) |
| 6 | 1:18 | 90 | 0.17 | 8 | 169 (83%) |

### Example 2

### Preparation of 1,1,2,2-tetramethoxyethane

The apparatus described under Example 1 was operated in such a way that 174 g of a mixture which consisted of 1.16 mol of glyoxal, 0.04 mol of 2,2-dimethoxyacetaldehyde, a small amount of p-toluenesulfonic acid and also of methanol and water were metered in at the top of the column. At the same time, gaseous methanol was introduced at the bottom of the column. The column was heated. On completion of metering, the bottom effluent was neutralized and worked up by means of vacuum distillation. 162 g (1.08 mol, 90% based on glyoxal and 2,2-dimethoxyacetaldehyde) of 1,1,2,2-tetramethoxyethane were isolated.

### Example 3

### Preparation of 1,1,2,2-tetraethoxyethane

The apparatus described under Example 1 was operated in such a way that a 40% aqueous glyoxal solution which had been admixed with a small amount of a solution of p-toluenesulfonic acid in ethanol was metered in at the top of the column at such a rate that the loading was 0.17 mol of glyoxal per hour. At the bottom of the column, gaseous ethanol was metered in at a rate of 2 mol/h. The column was heated. After a reaction time of 3 hours, the bottom effluent of the column which consisted of 1,1,2,2-tetraethoxyethane, ethanol and traces of 2,2-diethoxyacetaldehyde was worked up by distillation, and 65 g (0.32 mol; 62%) of 1,1,2,2-tetraethoxyethane could be isolated.

### Example 4

### Preparation of 1,1,2,2-tetrapropoxyethane

The apparatus described under Example 1 was operated in such a way that a 40% aqueous glyoxal solution which had been admixed with a small amount of a solution of p-toluenesulfonic acid in n-propanol was metered in at the top of the column at such a rate that the loading was 0.17 mol of glyoxal per hour. At the bottom of the column, gaseous n-propanol was metered in at a rate of 2 mol/h. The column was heated. After a reaction time of 2 hours, the bottom effluent of the column which consisted of 1,1,2,2-tetrapropoxyethane, n-propanol and approx. 1% of 2,2-dipropoxyacetaldehyde was worked up by means of vacuum distillation, and 48 g (0.19 mol; 54%) of 1,1,2,2-tetrapropoxyethane could be isolated.

### Example 5

### Preparation of 1,1,2,2-tetramethoxyethane

A 40% aqueous glyoxal solution was metered at a rate of 0.17 mol/h into the top of a heatable 1 m column (diameter 29 mm) filled with spheres of diameter 5 mm of the catalyst KA-3 (Sud-Chemie). At the same time, gaseous methanol was metered in at the bottom of the column. During the entire reaction, the apparatus was operated under nitrogen as a protective gas. The column was heated. A mixture of water and methanol was collected at the top of the column. The bottom effluent of the column consisted of a mixture of 1,1,2,2-tetramethoxyethane, 2,2-dimethoxyacetaldehyde, methyl 2-hydroxyacetate, 2-hydroxyacetic acid and methanol. The apparatus was operated for 4 hours, and the bottom effluent was analyzed by gas chromatography. According to the analysis, there were 11 g (0.07 mol, 11%) of 1,1,2,2-tetramethoxyethane.

### Comparative Example 1

### Preparation of 1,1,2,2-tetramethoxyethane

A 4 L four-neck flask equipped with dropping funnel, stirrer and a column in which the distillate could be removed via the top and via a sidestream was initially charged with 100 g of methanol, 1269 g (8.74 mol) of a 40% aqueous glyoxal solution and 70 g of p-toluenesulfonic acid. A mixture of water and methanol was distilled out of this mixture in such a way that water and methanol were withdrawn via the sidestream of the column and pure methanol was withdrawn overhead and fed back to the bottom. The amount of methanol withdrawn via the sidestream was determined by gas chromatography and replaced at the bottom by fresh methanol. After a reaction time of 11 hours, the formation of a mixture in the bottom consisting of 12.1% of 1,1,2,2-tetramethoxyethane, 2,2-dimethoxyacetaldehyde, water, methanol and some high-boiling components could be detected. It was possible to isolate 126 g of 1,1,2,2-tetramethoxyethane (0.84 mol, 10%) from this mixture by distillation.

The priority document of the present application, DE application 103.12 562.0, filed Mar. 21, 2003, is incorporated herein by reference.

Obviously, numerous modifications and variations on the present invention are possible in light of the above teachings. It is therefore to be understood that within the scope of the appended claims, the invention may be practiced otherwise than as specifically described herein.

What is claimed as new and is intended to be secured by Letters Patent is:

1. A process for preparing acetals of α,β-dicarbonyl compounds of the general formula (R″O)₂CRCR′(OR″)₂ (I), where
R and R′ are each independently H, (C₁-C₈)-alkyl, (C₃-C₈)-cycloalkyl, (C₂-C₈)-alkenyl, (C₂-C₈)-alkynyl or (C₆-C₁₈)-aryl and
R″ is in each case independently (C₁-C₈-alkyl), (C₃-C₈)-cycloalkyl, (C₂-C₈)-alkenyl or (C₂-C₈)-alkynyl or a chain X where X is a (C₂-C₁₂)-alkylene chain or (C₂-C₁₂)-alkenylene chain which joins the two oxygen atoms of the α-carbon atom and/or the two oxygen atoms of the β-carbon atom (acetals (Ia)) 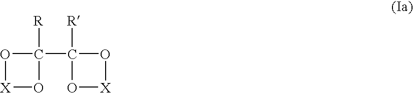
which comprises
continuously reacting compounds of the type R—CO—CO—R′ with alcohols of the type R″OH or HO—X—OH, where R, R′, R″ and X are each as defined above, in a countercurrent apparatus.

2. The process of claim 1, wherein the compound of the type R—CO—CO—R′ is fed into the countercurrent apparatus in liquid form or in the form of a solution and the compound R″OH or HO—X—OH is fed into the countercurrent apparatus in such a way that the vapor of the alcohol R″OH or HO—X—OH flows in countercurrent to the compound of the type R—CO—CO—R′ in liquid form or in the from of a solution.

3. The process of claim 1, wherein the compound R″OH or HO—X—OH is fed into the countercurrent apparatus in gaseous form in such a way that the compound R″OH or HO—X—OH in gaseous form flows in countercurrent to the compound of the type R—CO—CO—R′ in liquid form or in the form of a solution.

4. The process of claim 1, wherein the compound R—CO—CO—R′, in liquid form or as a solution, and the alcohol R″OH or HO—X—OH in liquid form are fed into the counter-current apparatus and the alcohol is vaporized in the apparatus in such a way that the vapor of the alcohol R″OH or HO—X—OH generated in this way flows in countercurrent to the liquid α,β-dicarbonyl compounds R—CO—CO—R′ or their solution.

5. The process of claim 1, wherein the countercurrent apparatus used is a thin-film evaporator, a falling-film evaporator, a reaction column or a stirred tank battery.

6. The process of claim 1, wherein the reaction is carried out in the absence or in the presence of an acidic or of a basic catalyst.

7. The process of claim 1, wherein the reaction is carried out in the presence of a homogeneous or heterogeneous catalyst.

8. The process of claim 1, wherein the reaction is carried out at a temperature of from 20 to 250° C.

9. The process of claim 1, wherein the reaction is carried out at a pressure of from 20 mbar to 20 bar.

10. The process of claim 1, wherein the molar ratio of the amount of α,β-dicarbonyl compound R—CO—CO—R′ fed in to the amount of the alcohol R″OH fed in is from 1:2 to 1:2000.

11. The process of claim 1, wherein the molar ratio of the amount of α,β-dicarbonyl compound R—CO—CO—R′ fed in to the amount of the alcohol R″OH fed in is from 1:8 to 1:20.

12. The process of claim 1, wherein the molar ratio of the amount of α,β-dicarbonyl compound R—CO—CO—R′ fed in to the amount of the alcohol HO—X—OH fed in is from 1:1 to 1:1000.

13. The process of claim 1, wherein the molar ratio of the amount of α,β-dicarbonyl compound R—CO—CO—R′ fed in to the amount of the alcohol fed in HO—X—OH is from 1:4 to 1:10.

14. The process of claim 1, wherein the secondary streams occurring in the reaction are isolated by means of distillation and recycled into the process.
